(19) United States
(12) Patent Application Publication
(10) Pub. No.: US 2023/0277080 A1
(43) Pub. Date: Sep. 7, 2023

(54) SOFT BIOSENSORS BASED ON GELATIN METHACRYLOYL (GelMA)

(71) Applicant: THE REGENTS OF THE UNIVERSITY OF CALIFORNIA, Oakland, CA (US)

(72) Inventors: Alireza Khademhosseini, Los Angeles, CA (US); Shiming Zhang, Los Angeles, CA (US); Zhikang Li, Los Angeles, CA (US)

(73) Assignee: THE REGENTS OF THE UNIVERSITY OF CALIFORNIA, Oakland, CA (US)

(21) Appl. No.: 18/040,115

(22) PCT Filed: Aug. 17, 2021
(86) PCT No.: PCT/US2021/046293

§ 371 Date: Jan. 31, 2023

Related U.S. Application Data
(60) Provisional application No. 63/067,021, filed on Aug. 18, 2020.

(51) Int. Cl. A61B 5/053; A61B 5/024; C08L 89/06; A61B 5/00

(52) U.S. Cl. CPC A61B 5/053; A61B 5/024; C08L 89/06; A61B 5/681; A61B 2562/12; A61B 2562/0247

(57) ABSTRACT

A gelatin methacryloyl (GelMA)-based biosensor device for wearable biosensing applications is disclosed. An exemplary capacitive tactile sensor with GelMA used as the core dielectric layer is disclosed. A robust chemical bonding and a reliable encapsulation approach are introduced to overcome detachment and water-evaporation issues in hydrogel biosensors. The resultant GelMA tactile sensor shows a high-pressure sensitivity of 0.19 kPa⁻¹ and one order of magnitude lower limit of detection (0.1 Pa) compared to previous hydrogel pressure sensors owing to its excellent mechanical and electrical properties (e.g., dielectric constant). Furthermore, it shows durability up to 3,000 test cycles because of tough chemical bonding, and long-term stability of three (3) days due to the inclusion of an encapsulation layer, which prevents water evaporation (e.g., 80% water content). Successful monitoring of various human physiological and motion signals demonstrates the potential of the GelMA biosensor device for wearable biosensing applications.

### RELATED APPLICATION

This application claims priority to U.S. Provisional Patent Application No. 63/067,021 filed on Aug. 18, 2020, which is hereby incorporated by reference. Priority is claimed pursuant to 35 U.S.C. § 119 and any other applicable statute.

### STATEMENT REGARDING FEDERALLY SPONSORED RESEARCH AND DEVELOPMENT

This invention was made with government support under Grant Numbers HL140951, GM126571, GM126831, and EB023052, awarded by the National Institutes of Health. The government has certain rights in the invention.

### TECHNICAL FIELD

The technical field relates to soft biosensors used for on-skin and in-vivo healthcare monitoring applications. More specifically, the technical field relates to tissue-compatible “soft” biosensor device based on a biodegradable hydrogel, gelatin methacryloyl (GelMA). In one embodiment, GelMA hydrogel was used as a dielectric layer in an electrical capacitor, but it can be easily used to make other kinds of soft sensors with other device architectures.

### BACKGROUND

Wearable soft tactile sensors have been in high demand in fast-growing fields, such as personalized healthcare, human-machine interfaces, and the internet of things because they allow real-time, low-cost, and long-term monitoring of human physiological signals. In the past decade, various wearable tactile sensors have been developed, including to monitor pressure, strain, vibration, temperature, and humidity. Among these sensors, pressure sensors are of great importance and widely investigated due to their ability to sense human physical and physiological signals such as gentle touch (<10 kPa), wrist pulse, blood pressure, heart rate, and respiration rate. However, to date, the majority of developed wearable pressure sensors are based on elastomers such as polydimethylsiloxane (PDMS), polyurethane (PU), polyethylene, and Ecoflex™ silicone rubbers. The mechanical mismatch between these elastomers (1 MPa˜1 GPa) and human tissue (˜10 kPa), as well as issues of biocompatibility, limits their future practical applications.

Compared to elastomers, hydrogels, consisting of three-dimensionally crosslinked polymers, are considered promising in developing next-generation wearable pressure sensors because of their intrinsic biocompatibility and extremely low Young's modulus. Therefore, increasing efforts are being devoted to developing hydrogel-based wearable pressure sensors. For instance, Yin et al., Micropatterned elastic ionic polyacrylamide hydrogel for low-voltage capacitive and organic thin-film transistor pressure sensors. Nano Energy, 2019, 58(96-104) discloses a polyacrylamide (PAAm) hydrogel pressure sensor based on the principle of electrical-double-layer (EDL), demonstrating a pressure sensitivity of 2.33 kPa⁻¹ in 0˜3 kPa. Huang J. et al. and Lei Z. et al. exploited polyvinyl alcohol (PVA) and polyacrylic acid (PAA)/alginate hydrogels based wearable ionic pressure sensors, respectively, showing corresponding pressure sensitivities of 0.033 kPa⁻¹ and 0.17 kPa⁻¹. See Huang J. et al., A Dual-Mode Wearable Sensor Based on Bacterial Cellulose Reinforced Hydrogels for Highly Sensitive Strain/Pressure Sensing, Advanced Electronic Materials, 2019, 6(1) and Lei Z. et al., A Bioinspired Mineral Hydrogel as a Self-Healable, Mechanically Adaptable Ionic Skin for Highly Sensitive Pressure Sensing, Advanced Materials, 2017, 29(22).

A wearable piezoresistive pressure sensor with pressure sensitivity of 0.05 kPa⁻¹ using a PVA-polyacrylamide (PAAm) hydrogel has also been demonstrated. Ge G. et al., Stretchable, Transparent, and Self-Patterned Hydrogel-Based Pressure Sensor for Human Motions Detection, Advanced Functional Materials, 2018, 28(32). Besides, various other hydrogels, such as polyacrylamide (PAAm) composite hydrogels, alginate composite hydrogels, gelatin hydrogels, and Fmoc-FF-PAni composite hydrogels, were also synthesized to construct wearable pressure sensors. These studies demonstrated the feasibility of using hydrogels in developing wearable pressure sensors. However, there are still unsolved challenges in hydrogel-based wearable biosensors, such as water evaporation, weak interface bonding, and the lack of cost-effective fabrication techniques for large-scale productions.

Gelatin methacryloyl (GelMA) is a hydrogel obtained by conjugating methacrylic anhydride (MA) to gelatin, which is derived directly from the skin. It has superior biocompatibility compared to other artificial hydrogels and has been widely used in cell culture, soft tissue adhesives, and implantations. Additionally, GelMA has the most similar Young's modulus to human tissue, which can contribute to excellent bio-mechanical matching at electronic-tissue interfaces. Its mechanical properties are also highly tunable, enabling GelMA-based devices to meet different mechanical stiffness requirements in practical applications. Importantly, GelMA has excellent robustness, allowing the recovery to its original shape after compressing. Furthermore, GelMA has good transparency, making it an ideal candidate for developing fully transparent bioelectronics. GelMA is a promising hydrogel for developing highly sensitive, skin-conformal, biocompatible, and transparent wearable pressure sensors.

### SUMMARY

In one embodiment, a GelMA-based wearable device is disclosed in the form of a capacitive tactile sensor for monitoring human physiological signals (FIG. 1). The electrical property (e.g., dielectric constant) of GelMA hydrogels was investigated under different synthesis conditions. Based on its tailorable mechanical and electrical properties, the feasibility of GelMA-based soft biosensors was demonstrated as a highly-sensitive and performance-tunable pressure sensor was developed with a layer-by-layer stacked capacitive assembly. Sequentially, a fully solution-processable and substantially optically transparent GelMA-based pressure sensor structure is proposed, with the conducting polymer PEDOT:PSS used as transparent electrodes and polydimethylsiloxane (PDMS)/GelMA/PDMS used as dielectric layers. In this unique design, the GelMA serves as the core dielectric layer while PDMS serves as an insulator between the GelMA dielectric layer and electrodes, and also as an encapsulation layer to prevent water evaporation of GelMA. A chemical bonding method is further introduced to solve the delamination issue between GelMA and elastomers. The resultant GelMA pressure sensor is structurally robust because of the enhanced interface bonding and fully transparent because of the optical transparency of each component.

Compared to previously reported hydrogel-based pressure sensors, the disclosed pressure sensor shows a higher sensitivity of 0.19 kPa⁻¹ and a lower (one order of magnitude) limit of detection (LOD) of 0.1 Pa. Furthermore, the GelMA pressure sensor shows high durability over 3,000 cyclic tests, and long-term stability up to 3 days of exposure to air, demonstrating the robustness of the device structure and the reliability of the encapsulation. Furthermore, the GelMA-based pressure sensor was successful in monitoring of human physiological signals, pulse, and vocal cord vibration using the developed GelMA-based hydrogel tactile sensors, illustrating their practical use in medical wearable applications.

GelMA hydrogel-based tactile sensors for medical wearable applications are disclosed herein. A unique and fully solution-processable device structure is disclosed using PDMS/GelMA/PDMS as dielectric layers and PEDOT:PSS as electrodes. This design includes four merits including: (1) fully solution-processable, which allows low-cost and large-area fabrication; (2) reduced water evaporation of the GelMA hydrogel by using a PDMS encapsulation layer; (3) improved stability and device reproducibility, because of the introduction of a tough bonding method with benzophenone; (4) transparency in the visible wavelength range. The GelMA hydrogel pressure sensors show a comparable pressure sensitivity of 0.19 kPa⁻¹, and a much lower LOD of 0.1 Pa (one order of magnitude) compared with those of previous hydrogel pressure sensors because of its excellent mechanical and electrical (dielectric constant) properties. It also shows excellent durability over 3,000 cycles because of the robust chemical bonding, and long-term performance stability up to 3 days of exposure to air because of the PDMS based encapsulation.

In one embodiment, a tissue-compatible biosensor device includes a first electrode comprising a biocompatible electrically conductive polymer; an inner insulation layer disposed on an inner side of the first electrode; a second electrode comprising a biocompatible electrically conductive polymer, the second electrode spaced apart from the first electrode to form a gap; an inner insulation layer disposed on an inner side of the second electrode; and crosslinked gelatin methacryloyl (GelMA) disposed in the gap between the inner side of the first electrode and the inner side of the second electrode. Both the first electrode and second electrode may also include respective outer insulation layers disposed on an outer surface of the first electrode and second electrode.

In another embodiment, a method of using the biosensor device includes disposing the biosensor device onto tissue and measuring a change in capacitance between the first electrode and the second electrode with a capacitance measurement device. The capacitance change may be used to sense and/or monitor one or more physiological parameters.

In another embodiment, a method of manufacturing a biosensor includes the operations of: providing a polydimethylsiloxane (PDMS) mold having a negative topology pattern formed thereon; filling the PDMS mold with gelatin methacryloyl (GelMA) precursor; laminating a first electrode structure to the filled PDMS mold and exposing the same to ultraviolet (UV) radiation to at least partially crosslink the GelMA and bond the first electrode structure; removing the GelMA and bonded first electrode structure from the mold; securing the removed GelMA and bonded first electrode structure to a second electrode structure; and exposing the secured structure (with first electrode and second electrode) to UV radiation.

### BRIEF DESCRIPTION OF THE DRAWINGS

FIG. 1A illustrates a side cross-sectional view of a GelMA hydrogel-based biosensor device according to one embodiment. In this embodiment the GelMA material forms microstructures between a first electrode and a second electrode.

FIG. 1B illustrates a side cross-sectional view of a GelMA hydrogel-based biosensor device according to another embodiment. In this embodiment the GelMA material forms a continuous layer between a first electrode and a second electrode.

FIG. 1C illustrates a schematic view of GelMA hydrogel-based capacitive tactile sensor used for medical wearable applications. GelMA hydrogel-based sensitive, biocompatible, and transparent tactile sensors are developed and used on the human body to real-time monitor various physiological signals for medical diagnostics. FIG. 1A shows a typical on-body use of GelMA hydrogel-based capacitive tactile sensors, including various human body-related physical motions (for example, vocal cord vibration and language recognition) and physiological signals (for example, wrist pulse and carotid artery pulse detection).

FIG. 1D illustrates a perspective view of an embodiment of a GelMA hydrogel-based capacitive tactile sensor. The GelMA hydrogel interior is illustrated showing microfeatures formed thereon.

FIG. 1E illustrates two illustrative graphs of capacitance change as a function of time showing the real-time detection of various physical motions (bottom) and physiological signals (top).

FIGS. 2A-2I illustrate various mechanical and electrical properties of GelMA hydrogels under different synthesis conditions. FIG. 2A illustrates the variation of the mechanical properties (storage modulus) with UV crosslinking time. FIG. 2B illustrates the variation of electrical property (relative permittivity) with UV crosslinking time. FIG. 2C shows the effects of MA volume (DS of MA) on the mechanical properties (storage modulus and loss modulus) of GelMA hydrogels. FIG. 2D shows the effects of MA volume (DS of MA) on the electrical property (relative permittivity) of GelMA hydrogels. FIG. 2E shows the effects of GelMA concentration on the mechanical properties (storage modulus and loss modulus) of GelMA hydrogels. FIG. 2F shows the effects of GelMA concentration of the electrical property (relative permittivity) of GelMA hydrogels. FIG. 2G shows the relative permittivity of GelMA hydrogels under different frequencies. FIG. 2H shows the comparison of Young's modulus of GelMA hydrogels with those of common elastomers (and tissue). FIG. 2I shows the comparison of the relative permittivity of GelMA hydrogels with those of common elastomers.

FIGS. 3A and 3B schematically illustrate stacked capacitive pressure sensors formed as a GelMA-based pressure sensor (FIG. 3A) or a PDMS-based pressure sensor (FIG. 3B). For both types of sensors, the aluminum film and parafilm are used as electrodes and insulation layers, respectively.

FIG. 3C shows a graph of the pressure sensitivity comparison between the two types of pressure sensors.

FIG. 3D shows a graph of the variation of pressure sensitivity with MA volume (i.e., DS of MA).

FIG. 3E shows a graph of the variation of pressure sensitivity with GelMA concentration.

FIG. 3F shows a graph of the capacitance changes of GelMA-based pressure sensors under different frequencies.

FIG. 4A shows the structural schematic of the designed GelMA hydrogel-based capacitive pressure sensor. The various layers that make the capacitive pressure sensor are seen in an exploded view (PDMS, PEDOT:PSS, GelMA, Cu—for leads).

FIG. 4B shows an embodiment of the fabrication process for forming the designed GelMA hydrogel-based capacitive pressure sensor of FIG. 4A including: i) Preparation of PDMS molds from a silicon wafer with pyramidal topology structure; ii) Immersing the PDMS mold into heated GelMA hydrogel precursor and sonicating to degas at 37° C.; iii) Laminating the PDMS/PEDOT:PSS/PDMS layer on the GelMA hydrogel precursor filled PDMS mold; iv) Partially UV crosslinking the GelMA hydrogel precursor to form micro pyramidal structure along with partially bonding the PDMS/PEDOT:PSS/PDMS layer with the microstructured GelMA hydrogel; v) Peeling off the PDMS/PEDOT:PSS/PDMS layer along with the microstructured GelMA hydrogel dielectric layer; vi) Zoom in view of the GelMA hydrogel dielectric layer; vii) Laminating another PDMS/PEDOT:PSS/PDMS layer with the microstructured GelMA hydrogel dielectric layer; viii) Exposing to UV light to completely crosslink the GelMA-based pyramidal structure as well as to toughly bond it with the top and bottom PDMS/PEDOT:PSS/PDMS layers, finishing the fabrication.

FIGS. 4C-4E illustrates photographic/SEM images of the GelMA-based pressure sensor, PDMS molds and silicon wafer with micro pyramidal structure. FIG. 4C is a digital photo of a fabricated GelMA-based pressure sensor. FIGS. 4D and 4E are SEM images of the GelMA hydrogel-based pyramidal structure and the silicon wafer.

FIGS. 5A-5G illustrates the performance evaluation of the fabricated microstructured GelMA hydrogel pressure sensor. FIG. 5A shows the pressure sensitivity in the pressure range of 0˜5 kPa, compared with that of the reference one based on a flat GelMA dielectric layer. FIG. 5B shows the capacitance response under small applied pressure. FIG. 5C (and with reference to Table 1) shows the comparison of pressure sensitivity and LOD of the GelMA hydrogel pressure sensor with previously reported hydrogel pressure sensors. FIG. 5D shows the capacitance response during loading and unloading process. FIG. 5E shows the response time tested with a mass of 100 mg. FIG. 5F shows the variation in pressure sensitivity with time at room temperature, from 10 hours to 72 hours. FIG. 5G illustrates the capacitance variation under a pressure of 0.5 kPa with more than 3,000 times of cyclic test.

FIG. 5H is a photo of the GelMA hydrogel pressure sensor with a printed badge of UCLA as background.

FIGS. 6A-6I shows exemplary applications of GelMA-based pressure sensors in monitoring of human physical or physiological signals. FIG. 6A illustrates the detection of air blowing from a nitrogen gun (e.g., simulating respiration of gases). FIG. 6B illustrates the sensing of finger touch force. FIG. 6C shows a schematic of human wrist pulse monitoring. FIG. 6D shows the results for wrist pulse detection. FIG. 6E is schematic illustration of the set up for carotid artery pulse measurement. FIG. 6F shows the capacitance change with carotid artery pulse. FIG. 6F is a schematic illustration of the set up for the vocal cord vibration detection. FIG. 6H shows the capacitance change when swallowing. FIG. 6I shows the capacitance variation when speaking letters, “U”-“C”-“L”-“A”.

FIGS. 7A-7C are photograph images showing robustness testing of GelMA hydrogels. FIG. 7A illustrates the original shape while FIG. 7B illustrates the shape during compression. FIG. 7C illustrates the shape after release. The GelMA hydrogel sample is highly compressible, and can recover to its original shape once released, indicating an excellent robustness.

FIGS. 8A-8C illustrates the theoretical analyses on the effect of thickness and Young's Modulus of the dielectric layer on pressure sensitivity of capacitive pressure sensors. FIG. 8A is a schematic representation of capacitive pressure sensors, where d₀, E₀ and εᵣ are the original thickness, Young's Modulus and relative permittivity of the dielectric layer, respectively; P is the applied pressure. FIG. 8B illustrates variation of pressure sensitivity with Young's Modulus E. FIG. 8C shows the variation of pressure sensitivity with the thickness d₀. The theories for these analyses are given by equations (1)-(3). FIG. 8B shows the change of pressure sensitivity with Young's Modulus, E, varying from E₀ to 120E₀ under the same pressure range (P, 0˜0.1E₀), where all results were calculated by combining equation (2) and equation (3). FIG. 8C shows the change of pressure sensitivity with the thickness of the dielectric layer, d₀, varying from 0.2d₀ to d₀ under the same pressure range (P, 0˜0.1E₀), where all results were calculated by combining equations (1) and (3).

FIGS. 9A-9B show the working mechanism of GelMA hydrogel-based capacitive tactile sensors. FIG. 9A is a schematic of the pressure sensor with the initial capacitance of C₀ and original dielectric layer thickness of d₀. FIG. 9B is a schematic of the pressure sensor under pressure, with reduced dielectric layer thickness (d) and increased capacitance (C). As shown in FIG. 9B, the working principle of the GelMA hydrogel-based pressure sensor is based on the capacitance variation induced by the applied pressure. The applied pressure compresses the middle GelMA dielectric layer, resulting in a reduced electrode distance and increased capacitance. The applied pressure can be quantitatively evaluated by the capacitance change. The microstructured dielectric layer is used to improve the pressure sensitivity by enhancing both its deformability and equivalent dielectric constant (including the air gap in the pyramidal structure) under pressure.

FIGS. 10A-10F illustrate a fabrication process used to make PDMS/PEDOT:PSS/PDMS film. FIG. 10A shows the spin-coating PDMS and curing at 80° C. for 2 hours. FIG. 10B illustrates the spin-coating PEDOT:PSS after plasma treatment of the PDMS surface and curing at 160° C. for 50 minutes. FIG. 10C illustrates the spin-coating PDMS and curing at 80° C. for 2 hours. FIG. 10D illustrates the operation of peeling off the PDMS/PEDOT:PSS/PDMS layer. FIG. 10E shows a magnified view of the PDMS/PEDOT:PSS/PDMS film. FIG. 10F illustrates the operation of cleaning the surface of the PDMS/PEDOT:PSS/PDMS layer with methanol, drying it with nitrogen gun, treating the insulation PDMS layer with benzophenone solution.

FIG. 11 illustrates the transmittance spectra of the GelMA hydrogel-based tactile sensor in the visible wavelength range of the electromagnetic radiation spectrum (from 400 to 760 nm).

FIGS. 12A-12E are images of the experimental GelMA-based pressure sensor used to monitor various physical and physiological signals. FIG. 12A shows an image of air blowing testing with a nitrogen gun. FIG. 12B is an image of gentle finger touch detection. FIG. 12C is an image of the GelMA-based pressure sensor being used to monitor wrist radial artery pulse monitoring. FIG. 12D illustrates the GelMA-based pressure sensor used for carotid artery pulse monitoring. FIG. 12E shows the GelMA-based pressure sensor used for the detection of swallowing and the vibration of vocal cord while speaking.

FIGS. 13A and 13B illustrate the experimental set-up for testing of the relative permittivity of GelMA hydrogels. FIG. 13A is a schematic representation of the set up used for the relative permittivity testing. Aluminum foils were used as top and bottom electrodes, and parafilm was used as insulators; the tᵢ and t_{d} represent the thicknesses of the GelMA layer and insulator, which are 1 mm and 0.085 mm, respectively; the dimension of each layer is the same: 20 mm×20 mm. FIG. 13B is an image of the set up used for the relative permittivity testing, where a thin glass is used to apply a certain pressure to compact the stacked multiple layers.

### DETAILED DESCRIPTION OF ILLUSTRATED EMBODIMENTS

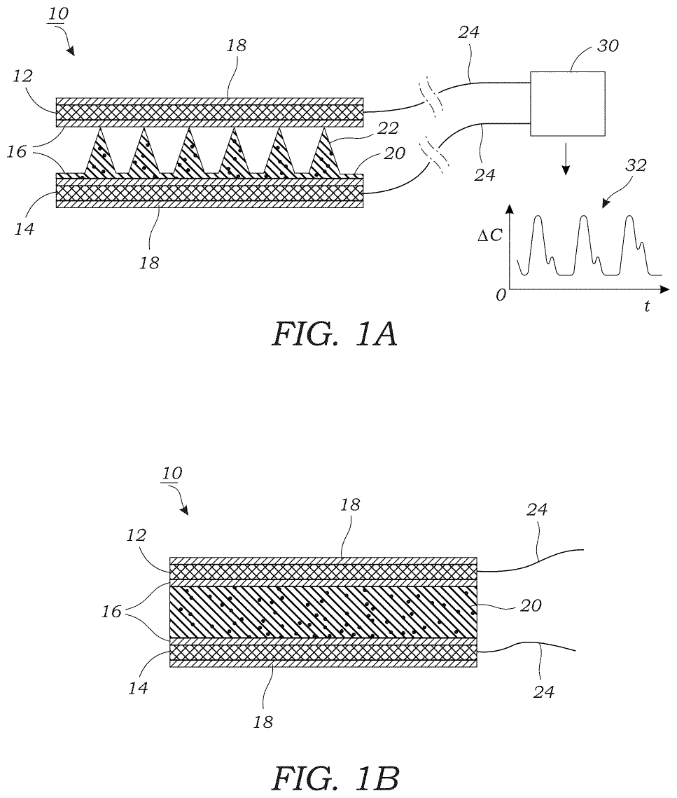

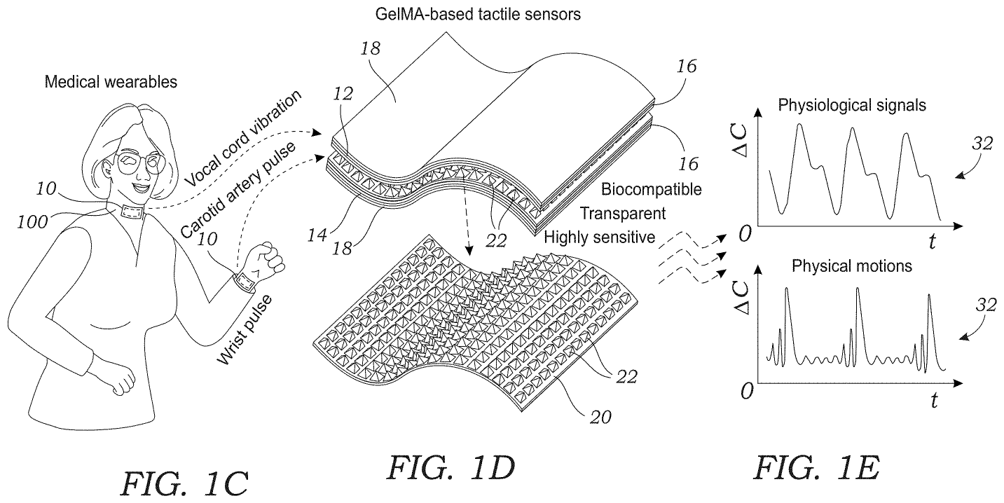

FIGS. 1A, 1B, 1D, 4, 9A, 9B illustrate embodiments of a tissue-compatible, soft biosensor device 10. The biosensor device 10 is soft in that it is flexible and can bend or flex. This enables the biosensor device 10 to be placed on a wide variety of surfaces including curved or irregularly shaped surfaces (e.g., skin tissue 100). Further, this enables the biosensor device 10 to remain in place for sensing and/or measurement even while undergoing flexing or bending forces. FIG. 1C, for example, shows the biosensor device 10 being used to sense and/or measure vocal cord vibration, carotid artery pulse, or wrist pulse. Multiple biosensor devices 10 may be used or only a single biosensor device 10 may be applied to tissue 100. The biosensor device 10 includes a first electrode 12 and a second electrode 14 that are made from a biocompatible, electrically conductive polymer. An example of such a polymer includes poly(3,4-ethylenedioxythiophene) polystyrene sulfonate (PEDOT:PSS). Both the first electrode 12 and the second electrode 14 have an insulation layer 16 formed on respective inner surfaces thereof. The outer surfaces of the first electrode 12 and the second electrode 14 may also include an insulation layer 18. In some embodiments, the inner insulation layer 16 and outer insulation layer 18 are made from the same material (e.g., polydimethylsiloxane (PDMS)). The insulation layers 16, 18 advantageously keep water from the hydrogel-based dielectric material from evaporating from the biosensor device 10.

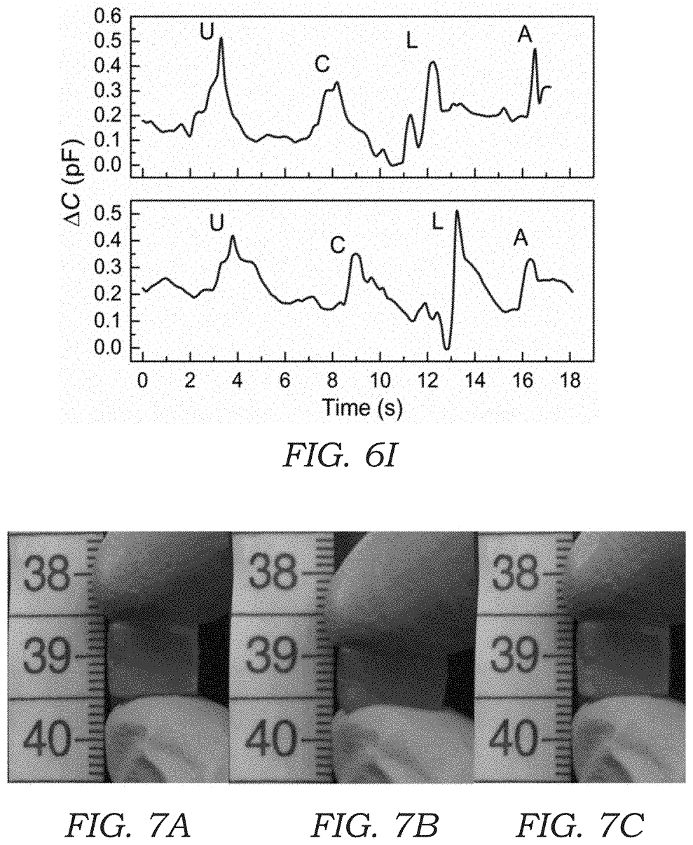

The first and second electrodes 12, 14 are spaced apart from one another by a gap that is filled with crosslinked gelatin methacryloyl (GelMA) material 20 in the form of a hydrogel which is compressible and can recover to its original shape once released (e.g., FIGS. 7A-7C). The GelMA material 20 may, in one embodiment, be located as a continuous layer within the gap (FIG. 1B). Alternatively, the GelMA material 20 inside the gap may be formed as a plurality of microstructures 22 such as illustrated in FIGS. 1A, 1D, 4A, 4B, 4D, 9A, 9B. The microstructures 22 are protuberances or surface features that extend away from a base or substrate. In between the microstructures 22 are gaps or voids with no GelMA material 20. As explained herein, sensitivity of the biosensor device 10 is improved by using GelMA microstructures 22. The microstructures 22 may include pyramidal or needle-like structures (e.g., microneedles) although other shapes and geometries may be used. Additional examples include hemispherical, cylindrical, post, fin, grate-shaped microstructures 22. The microstructures 22 generally have a height that is less than 750 μm in an uncompressed state. The microfeatures 22 extend across the gap formed between the first electrode 12 and the second electrode 24 to create or form the dielectric layer. Wires 24 are connected to the first and second electrodes 12, 14. To use the biosensor device 10, a capacitance measuring device 30 is secured to the first and second electrodes 12, 14 via the wires 24. The capacitance measuring device 30 may include a standard LCR meter or capacitance measuring circuitry contained within a device that is used to read capacitance values and/or capacitance changes. This may include a portable electronic device. FIGS. 1A and 1E illustrates an example of the change in capacitance signal 32 as measured by the capacitance measuring device 30 as function of time. The change in capacitance may be indicative of a physiological signal or motion (e.g., pulse, breathing, touch, swallowing, talking, etc.).

In some embodiments, the capacitance measuring device 30 may be co-located with or near the biosensor device 10. For example, a user may also wear capacitance measuring device 30 that is electrically connected to the biosensor device 10. Alternatively, the capacitance measuring device 30 may be a small electronic device that can be kept in the home or medical office/hospital setting and connected to biosensor device 10 via wires 24. The capacitance measuring device 30 may transmit data wirelesses (e.g., using WiFi, Bluetooth, etc.) to another computing device for viewing and/or analysis of the generated data. For example, data may be transmitted wirelessly to a local or remote computer (e.g., server) which can be viewed by the user or other healthcare professional. The capacitance measuring device 30 may also locally store capacitance signal 32 data in a memory or the like. This data can then be transmitted or downloaded/offloaded periodically to a local or remote computer.

The biosensor device 10 may optionally include an adhesive formed on one side thereof so that the biosensor 10 can be secured to the sensing surface such as tissue 100. The optional adhesive may be formed on the first electrode 12, second electrode 14, or the insulation layer(s) 18. One or more fasteners may also be used to secure the biosensor device 10 to the tissue 100. This may include a band, bandage, wrap, or the like. The sensing surface, in one embodiment, is living tissue 100 of a mammal. This may include, for example, skin surfaces although it may be placed on other organ tissues 100. In one preferred embodiment, the biosensor device 10 is placed on an external skin surface of the subject. The biosensor device 10 may be used to measure a number of physiological parameters such as, for example, pulse/pulse rate, respiration/respiration rate, blood pressure, swallowing, voice (e.g., sound from vocal cords), bodily sounds, and touch/physical pressure.

In one particular embodiment, the biosensor device 10 is made from a first electrode 12 and a second electrode 14 that includes crosslinked GelMA material 20 located in the gap formed between the first and second electrodes 12, 14. Each electrode, 12, 14 is surrounded by an outer insulation layer 18 and an inner insulation layer 16 made from polydimethylsiloxane (PDMS) while the electrodes are formed from poly(3,4-ethylenedioxythiophene) polystyrene sulfonate (PEDOT:PSS) (i.e., forming a PDMS/PEDOT:PSS/PDMS structure). Wires 24 or other conductors are connected to the PEDOT:PSS electrodes 12, 14. In an alternative embodiment, the inner insulation layer 16 and/or the outer insulation layer 18 may be made from a degradable material. An example includes biodegradable proteins.

### EXPERIMENTAL

Mechanical Properties and Relative Permittivity Characterization of GelMA Hydrogels

Material properties of the dielectric layer, such as Young's modulus, viscous modulus, and relative permittivity, are key parameters influencing the performances of capacitive pressure sensors. For GelMA hydrogels, their mechanical property and relative permittivity are dependent on several variables: the degree of substitution (DS) of MA, the concentration of GelMA, the concentration of photoinitiator (PI), UV light strength and UV crosslinking time. To date, the effects of these parameters on their viscous modulus and electrical permittivity have been rarely investigated, which are directly related to the response time and pressure sensitivity of the capacitive pressure sensor, respectively. The dependence of modulus and relative permittivity of GelMA hydrogels with respect to UV crosslinking time was first studied by fixing the other variables. That was to determine the time required for complete chemical crosslinking and to obtain stable mechanical and electrical properties. Subsequently, the dependence on MA volume (DS of MA) and GelMA concentration was investigated at the determined crosslinking conditions (PI concentration, UV light strength, and crosslinking time), revealing the tailorable range of the modulus and relative permittivity.

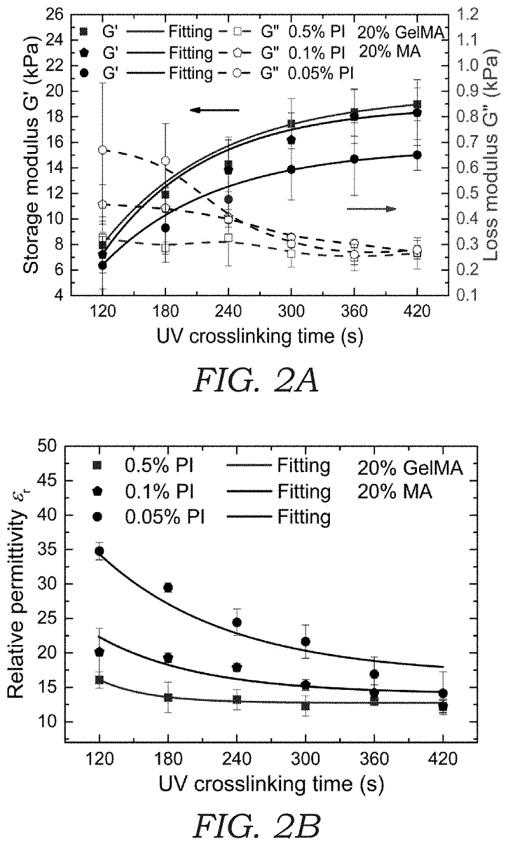

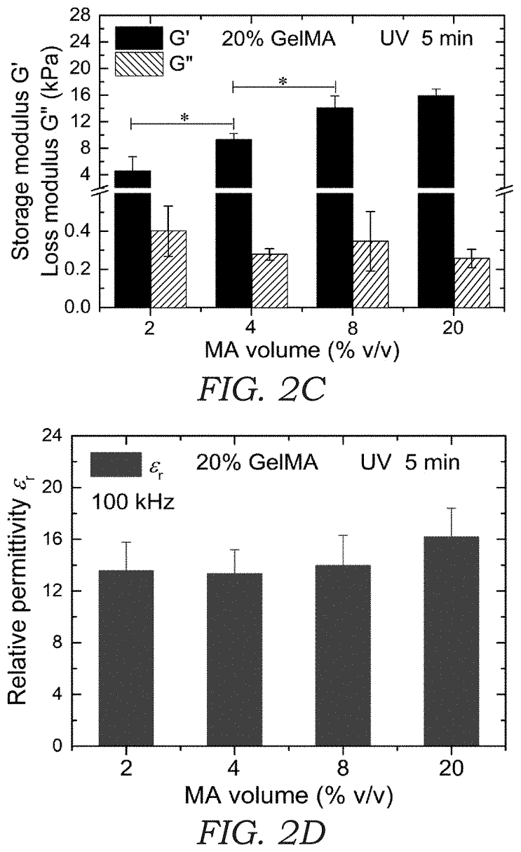

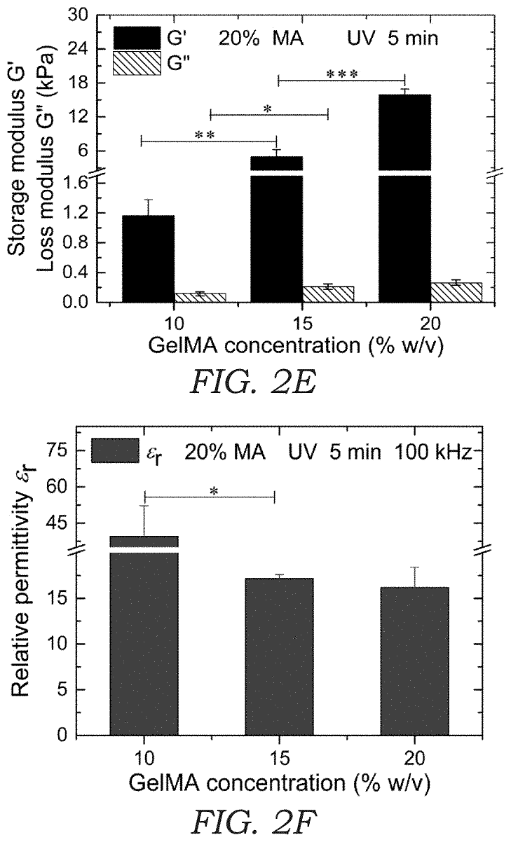

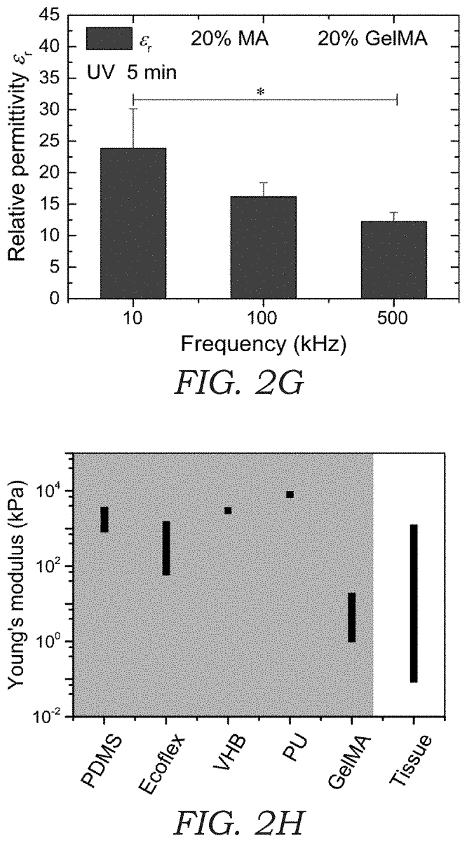

The results of the mechanical and electrical properties of the GelMA hydrogels under different synthesis conditions are shown in FIGS. 2A-2F. The storage modulus (both loss modulus and relative permittivity) increased (decreased) with time (<300 s) and reached a plateau afterward (>300s) for different PI concentrations of GelMA hydrogels, indicating the chemical crosslinking was completed over 300 seconds (FIGS. 2A and 2B). In the following experiments, the effects of MA volume (DS of MA) and GelMA concentration were investigated on both properties of the GelMA hydrogels. The storage modulus of the GelMA hydrogels was observed to increase from 4.6 kPa to 15.9 kPa when increasing the MA volume from 2% to 20% (FIG. 2C), and from 1.2 kPa to 15.9 kPa when increasing the GelMA concentration from 10% to 20% (FIG. 2E). The loss modulus showed no obvious dependence on MA volume and GelMA concentration (˜0.3 kPa in FIG. 2C) and ˜0.2 kPa in FIG. 2E), indicating their neglected influence on the response time. The relative permittivity showed a negligible change with increased MA volume, but a distinct decrease (39˜16) with increased GelMA concentrations (10%˜20%), which could be attributed to the lower water content in the highly crosslinked GelMA hydrogel (FIGS. 2D and 2F). The relative permittivity was also significantly affected by the increased frequency, i.e., 24 at 10 kHz and 12 at 500 kHz (FIG. 2G). Both Young's modulus (1.2˜15.9 kPa) and relative permittivity (16˜39) of GelMA hydrogels could be tailored in a wide range, highlighting their superior tunability. The Young's modulus of GelMA hydrogels is two orders of magnitude lower (FIG. 2H), and their electrical permittivity is three times higher (FIG. 2I) than those of their elastomeric competitors. These outstanding properties indicate the great potential of GelMA hydrogels for use as the GelMA material 20 in developing high-performance biosensor devices 10 (e.g., capacitive pressure sensors).

Performance and Tunability Evaluation of GelMA Hydrogels for Capacitive Pressure Sensors

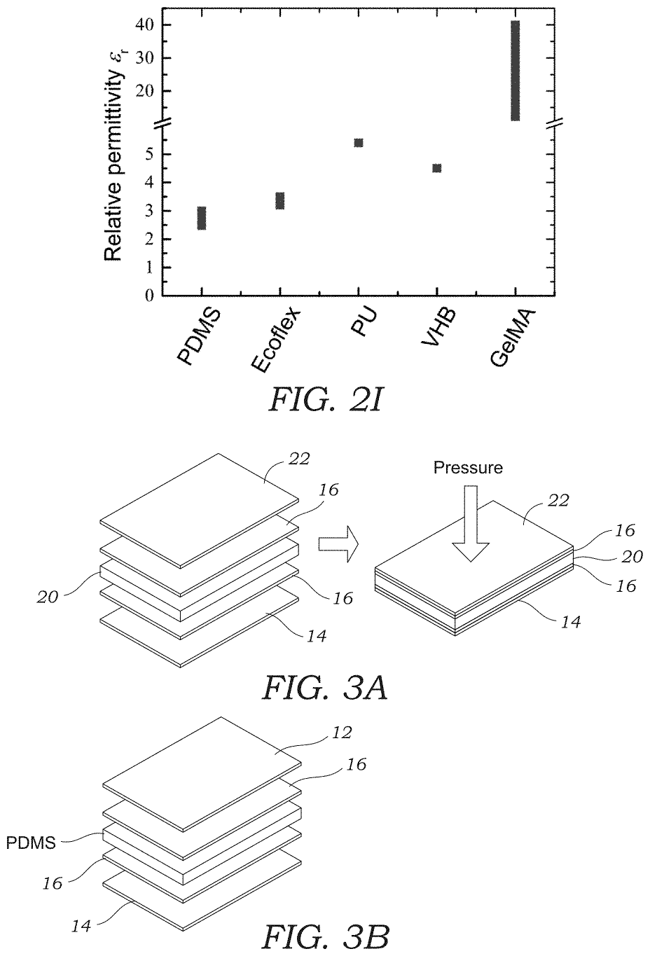

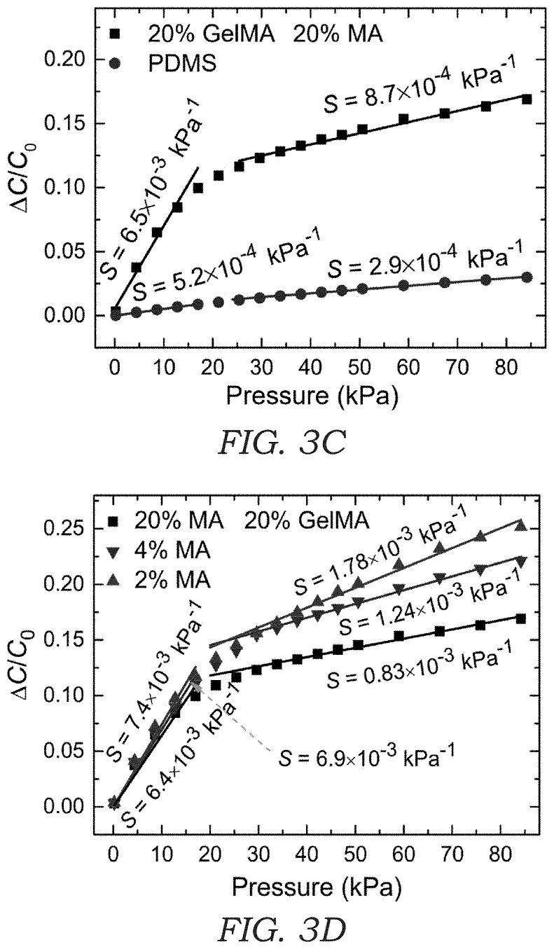

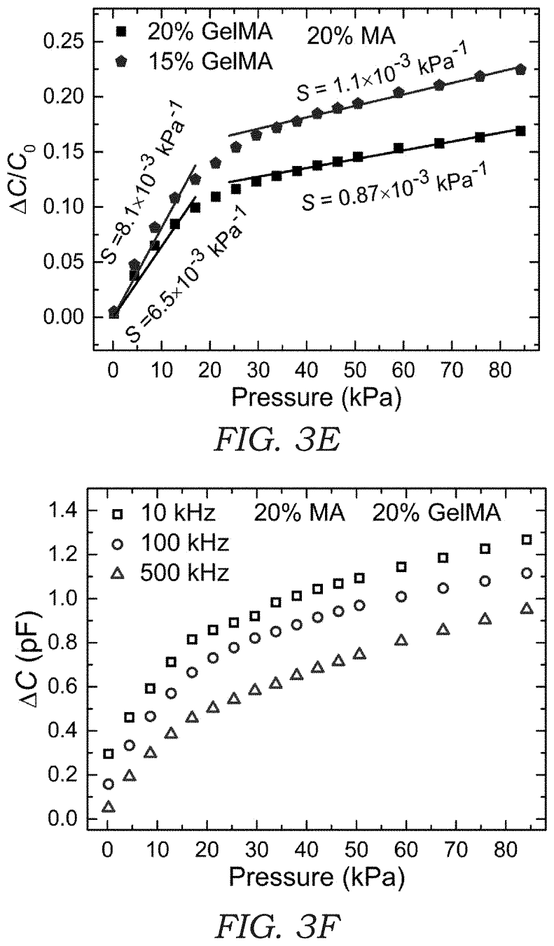

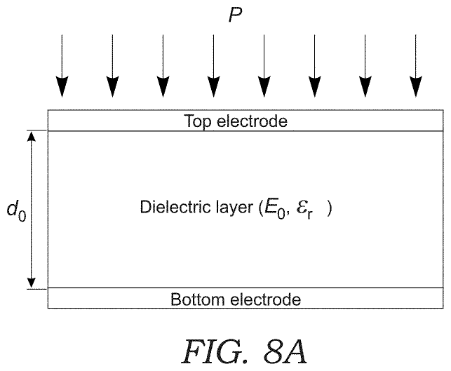

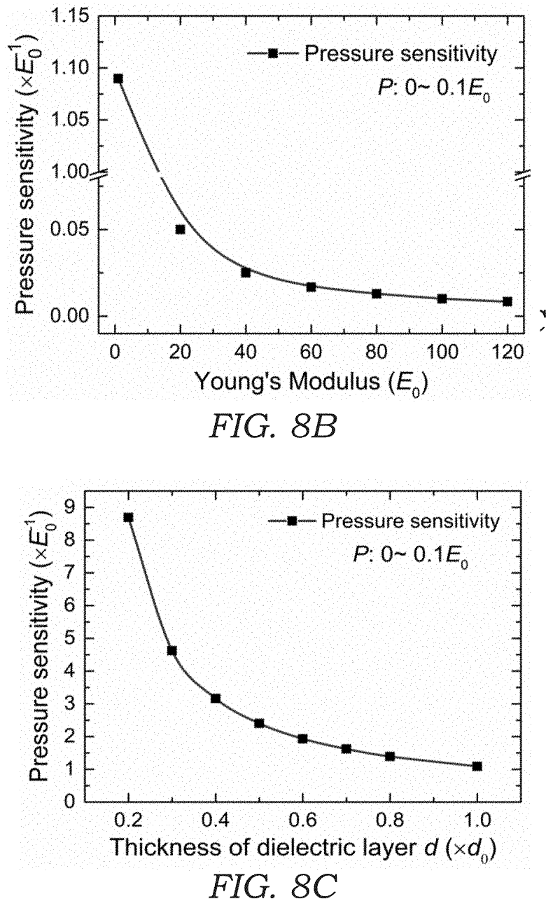

To evaluate the potential of using GelMA hydrogels to develop pressure sensors, a biosensor device 10 in the form of a capacitive pressure sensor was assembled with a layer-by-layer stacking method (FIG. 3A). The device performance was compared to that of a reference device fabricated with PDMS as the dielectric layer in a pressure range between 0 and 84 kPa (FIG. 3B). As shown in FIG. 3C, the stacked GelMA-based pressure sensor 10 showed a pressure sensitivity of 6.5×10⁻³ kPa⁻¹ in the pressure range of 0˜17 kPa, which was one order of magnitude higher than that of the PDMS-based device. The sensitivity decreased to 8.7×10⁻⁴ kPa⁻¹ in the pressure range of 25 kPa˜84 kPa, which was attributable to the reduced deformation ability of GelMA hydrogels under high pressure. These results indicate that, compared with elastomers, GelMA hydrogels enable a significant improvement in pressure sensitivity. The pressure sensitivity can be further increased to approach its theoretical limit (two orders of magnitude higher than that of PDMS-based pressure sensors, shown in FIG. 8B) by optimizing the stacked pressure sensor and experimental testing approach. FIG. 8A is a schematic representation of capacitive pressure sensors, where d₀, E₀ and εᵣ are the original thickness, Young's Modulus and relative permittivity of the dielectric layer, respectively; P is the applied pressure.

The sensitivity of GelMA pressure sensors 10 can be tuned by changing the MA volume and GelMA concentrations. The pressure sensitivity can be increased with decreased MA volume and GelMA concentrations, which is reasonable because of the decrease in Young's modulus. For the studied case, a 114% (from 0.83×10⁻³ to 1.78×10⁻³) increase in pressure sensitivity was observed when the volume ratio of MA reduced from 20% to 2% (FIG. 3D). A 40% (from 6.5×10⁻³ to 8.1×10⁻³) increase was observed when the GelMA concentration decreased from 20% to 15% (FIG. 3E). This tunability can be significantly amplified when the thickness of the GelMA dielectric layers is reduced, which is theoretically demonstrated in FIG. 8C. Consistent with the change in relative permittivity, the capacitance variation of the GelMA pressure sensor 10 increased with decreased frequency (FIG. 3F), suggesting another approach to tune the pressure sensitivity. Taken together, the pressure sensitivity of GelMA hydrogel-based biosensor device 10 can be tuned by varying the MA volume, GelMA concentration, and/or working frequency.

Enabling all Solution-Processed Pressure Sensors with Microstructured GelMA Hydrogels

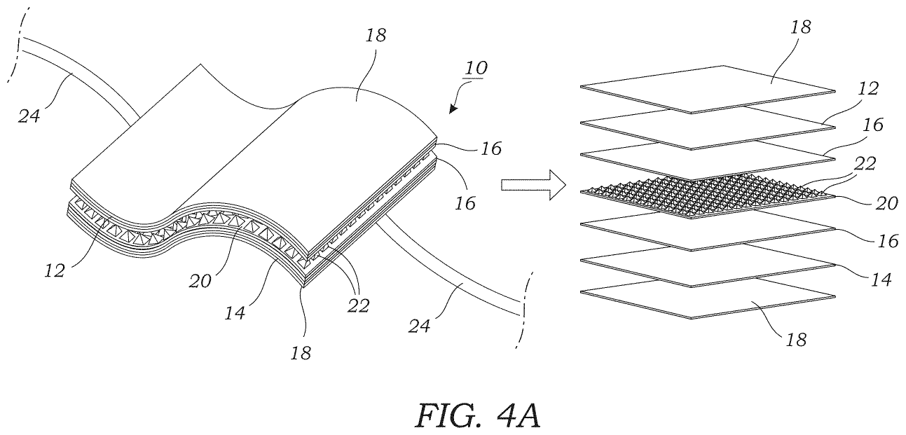

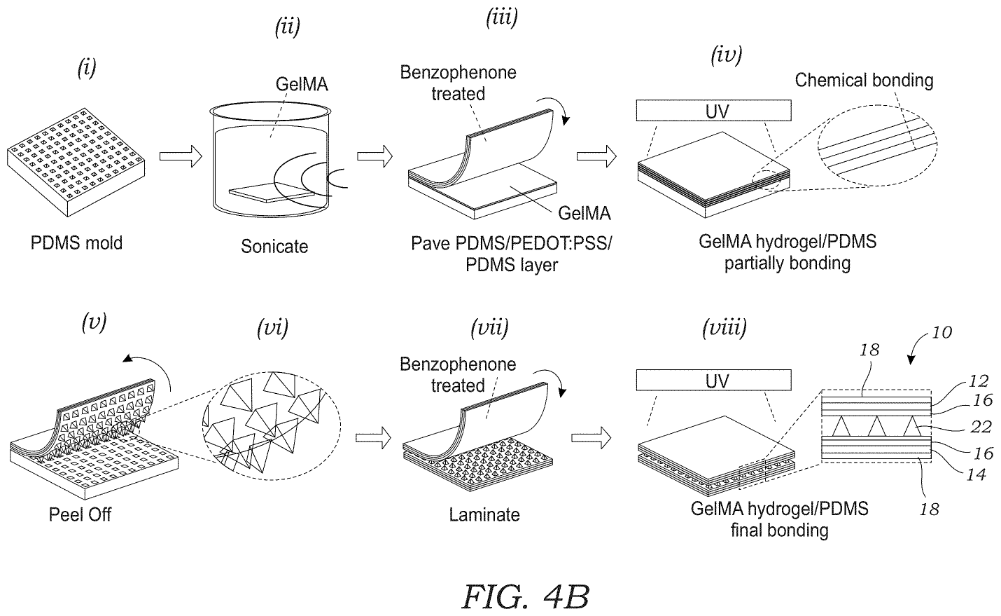

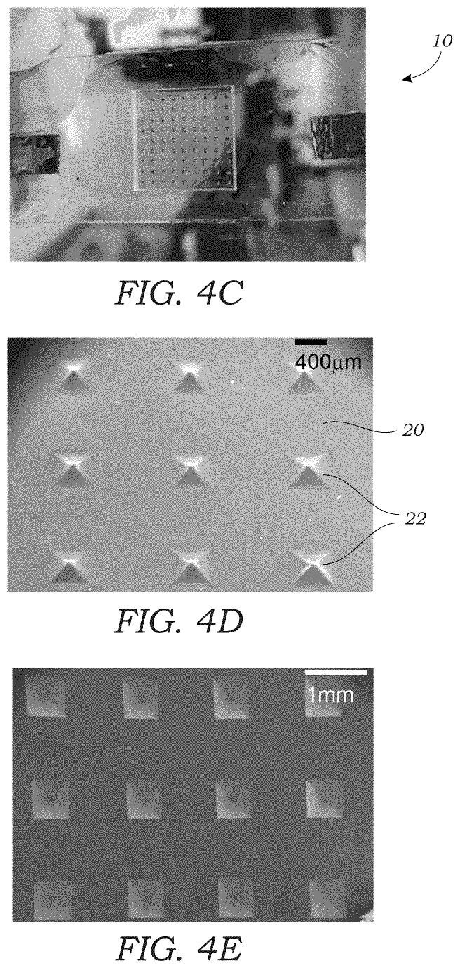

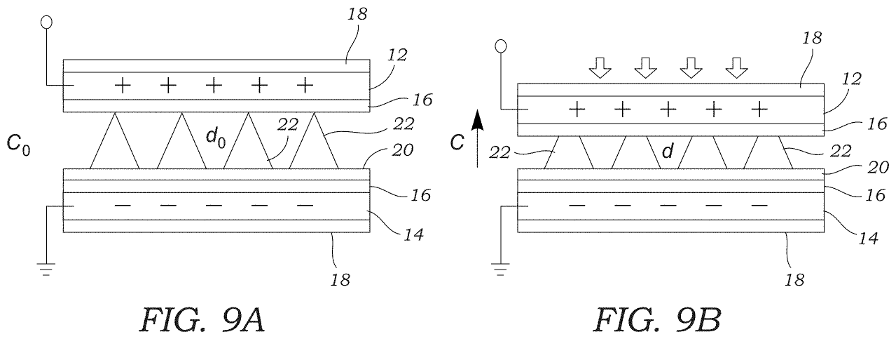

As described above, a layer-by-layer stacked structure was used to evaluate the ability of GelMA hydrogels as the GelMA material 20 in developing highly-sensitive and performance-tunable biosensor devices 10 that function as pressure sensors. Further development of the solution-processable fabrication technique could take full advantage of the GelMA hydrogel for low-cost and large-area production. Towards this goal, a unique device structure was then investigated where PDMS/GelMA/PDMS is used as a dielectric layer (formed by insulation layers 16 and GelMA material 20 located in gap), and PEDOT:PSS as electrodes 12, 14 (FIG. 4A). PEDOT:PSS is selected because of its solution processability, high conductivity and transparency. PDMS is employed for the insulating layers 16 because of its biocompatible compliance with human tissue, solution-processability, and much lower Young's modulus than that of the most-often used substrate material, polyethylene terephthalate (PET) (E, ˜2.7 GPa). GelMA material 20 is used as the core dielectric layer, and is made into pyramidal microstructures 22 to improve the pressure sensitivity. The PDMS layer 16 interposed between the GelMA material 20 and PEDOT:PSS layers of the electrodes 12, 14 has three functions: (1) acts as insulator layer to prevent ionic conduction between the electrode 12, 14 and GelMA material 20 and increases the device reproducibility; (2) serves as an encapsulation layer to avoid the water-loss of the GelMA hydrogel material 20; and (3) allows tough interface adhesion between PDMS and GelMA when pre-treated with benzophenone solution. Finally, together with the interposed PDMS layer 16, an outmost PDMS substrate layer 18 is used to prevent possible delamination during long-term operation and conductivity deterioration of the PEDOT:PSS electrodes 12, 14 in air and humid conditions. It should be noted that the interposed PDMS layer 16 has a negligible effect on the device 10 performance. The working mechanism of the microstructured GelMA pressure sensor 10 is illustrated in FIGS. 9A-9B.

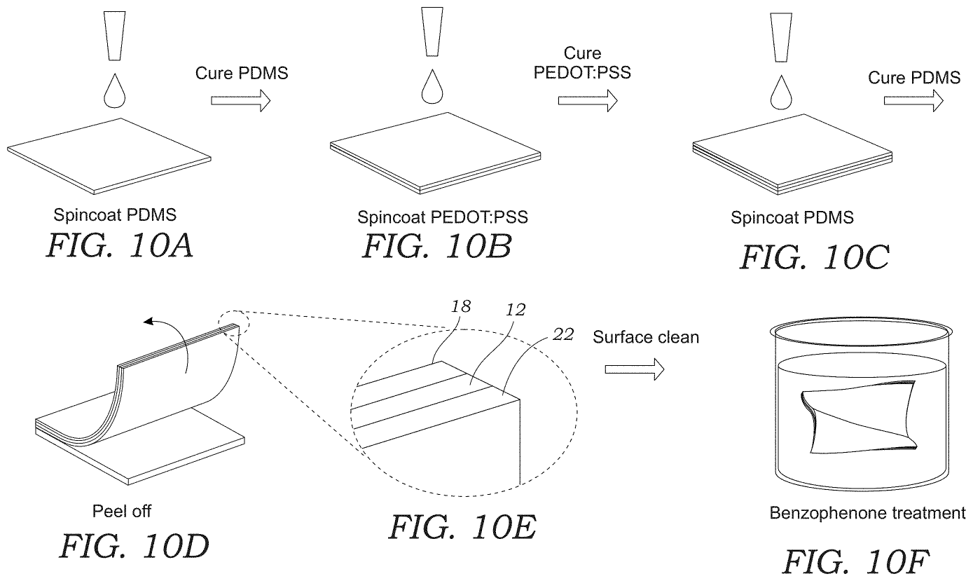

The fabrication process of the biosensor device 10 in the form of a microstructured GelMA hydrogel pressure sensor began with the preparation of a PDMS mold from a silicon wafer with a pyramidal topology structure (FIG. 4B(i)). Sequentially, the PDMS mold was immersed into a pre-heated GelMA precursor solution, followed by sonication at 37° C. to remove bubbles in the pyramidal cavities of the PDMS mold (FIG. 4B(ii)). Next, the PDMS mold with filled GelMA precursor was laminated to a benzophenone-treated PDMS/PEDOT: PSS/PDMS film (see FIGS. 10A-10F) under pressure, improving the structure uniformity (FIG. 4B(iii)). The GelMA precursor filled PDMS mold was then exposed to UV light to partially crosslink GelMA hydrogel, and simultaneously bond the GelMA hydrogel with the PDMS/PEDOT:PSS/PDMS electrode 12, 14 (FIG. 4B(iv)). This partial crosslinking aims to increase the adhesion of the microstructured GelMA hydrogel material 20 with the other electrode 12, 14. The mechanism of the bonding between PDMS and GelMA is based on the alleviation of oxygen inhibition effect and surface activation of PDMS after the treatment of benzophenone solution. The stretchy polymer networks of the pre-shaped GelMA hydrogels are grafted on PDMS by covalent crosslinking under UV light exposure, resulting in a robust interface. The bonded electrode/GelMA film was peeled off (FIG. 4B(v)) from the PDMS mold and attached to the second benzophenone-treated PDMS/PEDOT:PSS/PDMS electrode 12, 14 (FIG. 4B(vii)). The device 10 fabrication was completed with a final exposure to UV light, fully crosslinking the microstructured GelMA hydrogel material 20 and increasing its bonding with both electrodes 12, 14 (FIG. 4B(viii)). The GelMA hydrogel pressure sensor devices 10 fabricated using the fully solution-based process is shown in the images of FIG. 4C-4E, where the microstructured GelMA hydrogel dielectric layer and silicon mold are illustrated in FIGS. 4D and 4E, respectively.

Performance of all Solution-Processed and Microstructured GelMA Hydrogel Pressure Sensors

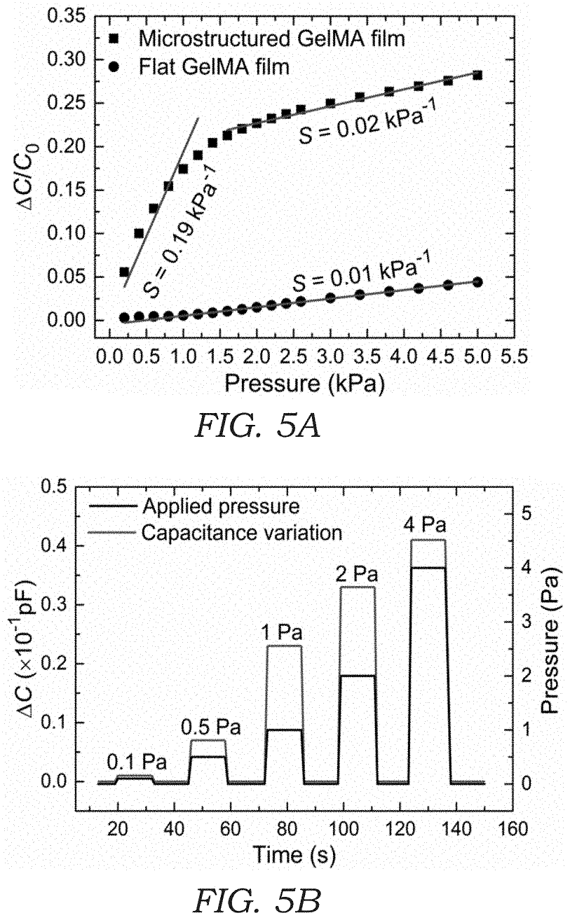

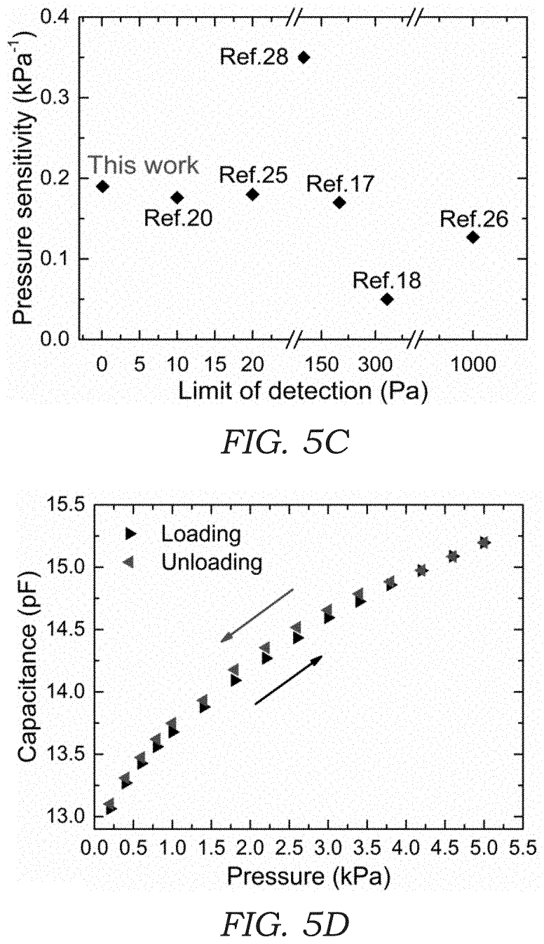

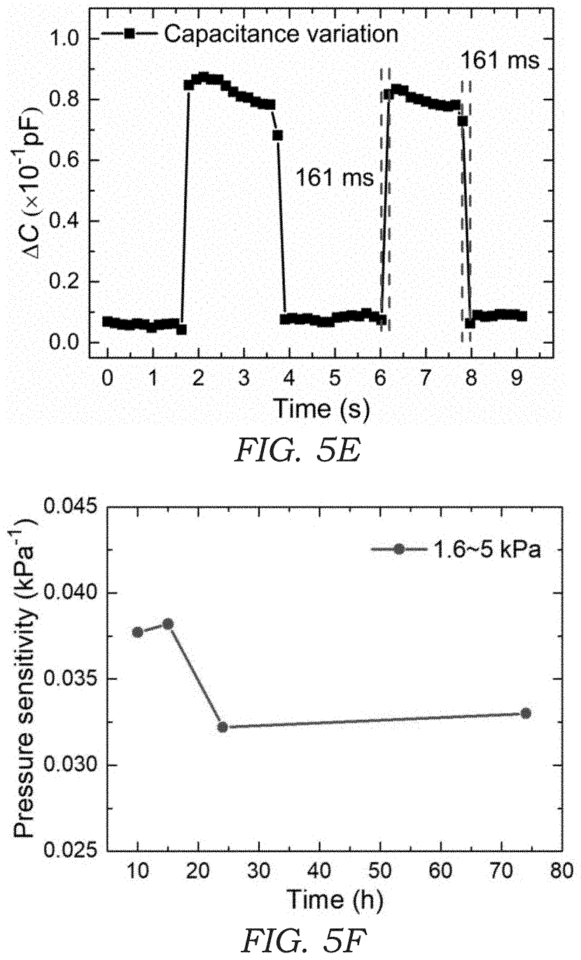

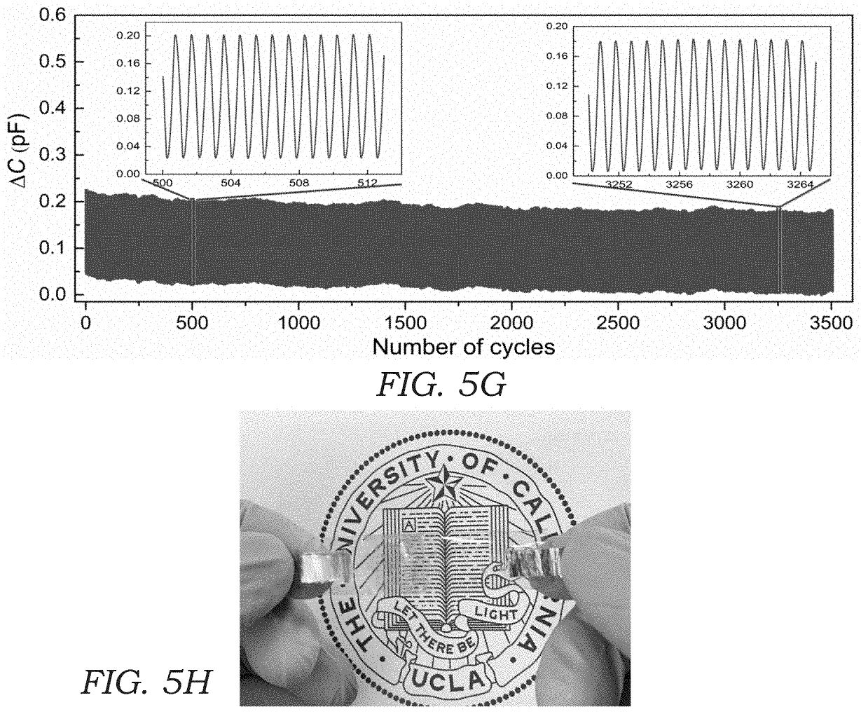

FIGS. 5A-5G shows the performance of the all-solution processed hydrogel pressure sensor 10 with microstructured GelMA hydrogel material 20 dielectric layers, which have a height of 420 μm, base width of 600 μm, and spacing of 1 mm with a water content of 80%. The biosensor device 10 was tested between 0˜5 kPa, which covers the pressure range of human physiological motions such as wrist pulse and vocal cord vibration. As shown in FIG. 5A, a pressure sensitivity of 0.19 kPa⁻¹ was obtained between 0 and 1.2 kPa. The pressure sensitivity was 19 times higher than that of the reference device with a flat GelMA hydrogel dielectric layer. It decreased to 0.02 kPa⁻¹ when the pressure increased from 1.6 kPa to 5 kPa, attributable to a reduced deformation ability of the GelMA hydrogel material 10 dielectric layer under high pressure. The LOD of the pressure sensor 10 reached as low as 0.1 Pa, which was tested with small weights of 1 mg, 5 mg, 10 mg, 20 mg, and 40 mg (corresponding to 0.1 Pa, 0.5 Pa, 1 Pa, 2 Pa and 4 Pa) (FIG. 5B). The pressure sensitivity of the GelMA hydrogel pressure sensor 10 is higher, while its LOD is one order of magnitude lower than that of previously reported hydrogel pressure sensors (FIG. 5C and Table 1). The pressure sensitivity could be further improved by tuning the size and arrangement of the pyramid-shaped microstructures 22 (see Table 2 for a list of commonly used dimensions of previously reported pyramid structures).

Hysteresis is one of the factors determining the accuracy of pressure sensors, which is related to the viscoelasticity of the dielectric materials. The GelMA pressure sensors 10 show minimal signal variance because of the low loss modulus of GelMA hydrogel. The hysteresis was tested in the pressure range of 0 to 5 kPa analogous to wrist pulse and vocal cord vibration pressure. As shown in FIG. 5D, a small hysteresis error of 4% was observed, which was much lower than that in previous reports. The biosensor device 10 showed a fast response time of ˜161 ms, both in the pressurization and relaxation process, with a weight of 100 mg (corresponding to a pressure of 10 Pa) (FIG. 5E). To further evaluate the potential of the GelMA pressure sensor 10 for practical applications, its long-term stability was investigated by exposing the biosensor device 10 to air for three (3) days. FIG. 5F shows the pressure sensitivity tested at different times (10 h, 15 h, 24 h, and 72 h). The pressure sensitivity shows a minor change of 12% (i.e., from 0.037 to 0.033 kPa⁻¹), attributable to the unique design of the PDMS/GelMA/PDMS dielectric layer where the secondary PDMS encapsulation layer 18 could efficiently prevent the water evaporation of the GelMA hydrogel. Furthermore, the biosensor device 10 was tested for more than 3,000 compression/release cycles (0-0.5 kPa), showing negligible capacitance reduction and no delamination between the GelMA-based core dielectric layer and PDMS encapsulation layer 16 (FIG. 5G). The excellent durability reflects the robustness of the chemical bonding between the PDMS and GelMA hydrogel material 20 that forms the pressure sensor 10.

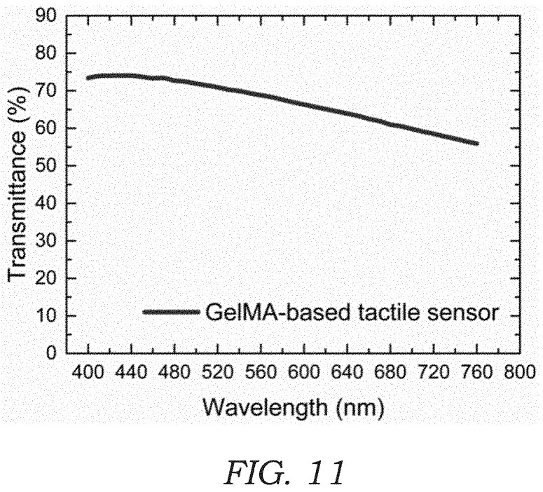

In addition, the GelMA pressure sensors 10 are substantially optically transparent (FIG. 5H), showing a transmittance of ˜69% at 550 nm wavelength (FIG. 11). This is because, during the fabrication of the biosensor device 10, all the materials selected, GelMA, PDMS, as well as the electrode PEDOT:PSS, are optically transparent. A fully transparent pressure sensor 10 is of importance in enabling invisible wearable applications. To summarize, the small hysteresis, fast response, excellent durability, and good transparency co-demonstrate the excellence of the GelMA pressure sensor 10 for practical wearable applications.

Applications of GelMA Hydrogel Tactile Sensors in Medical Wearables

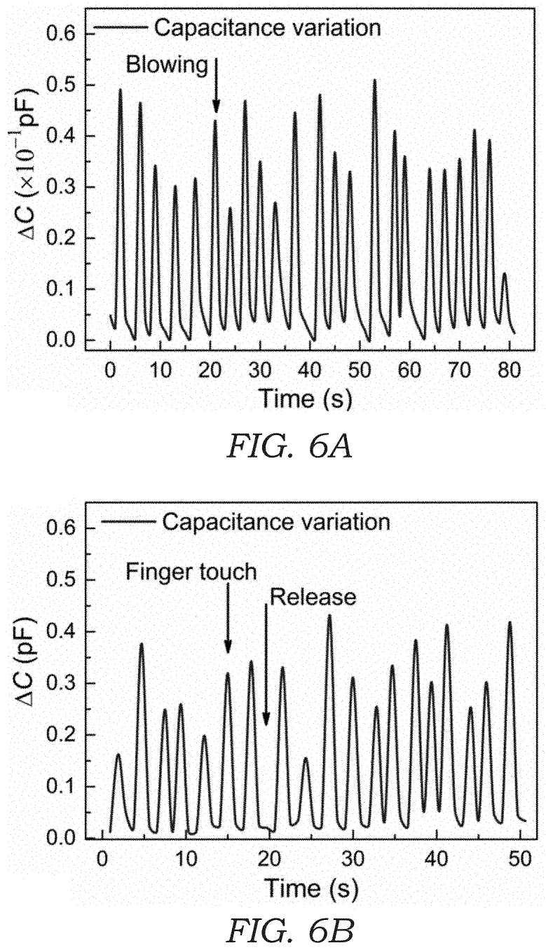

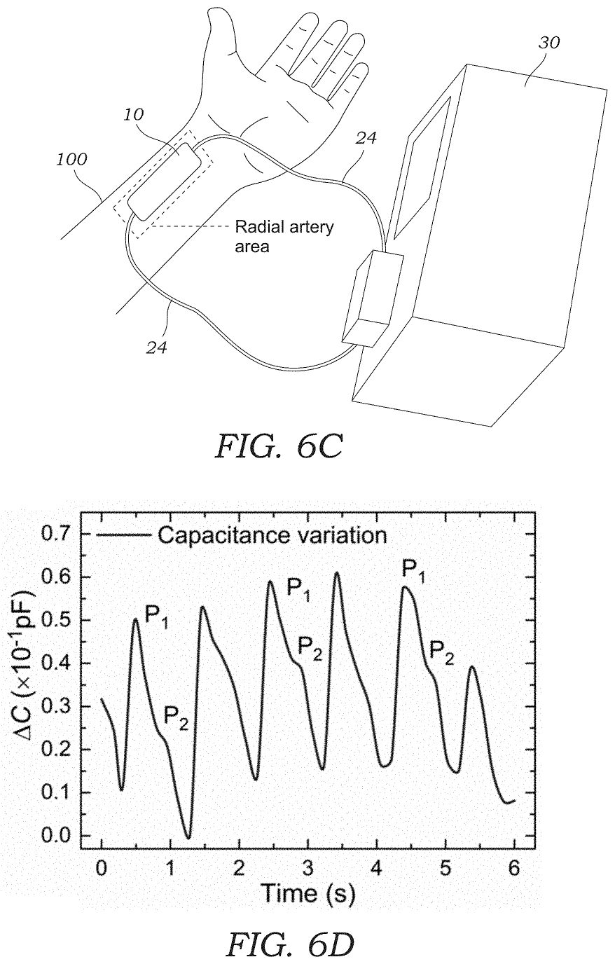

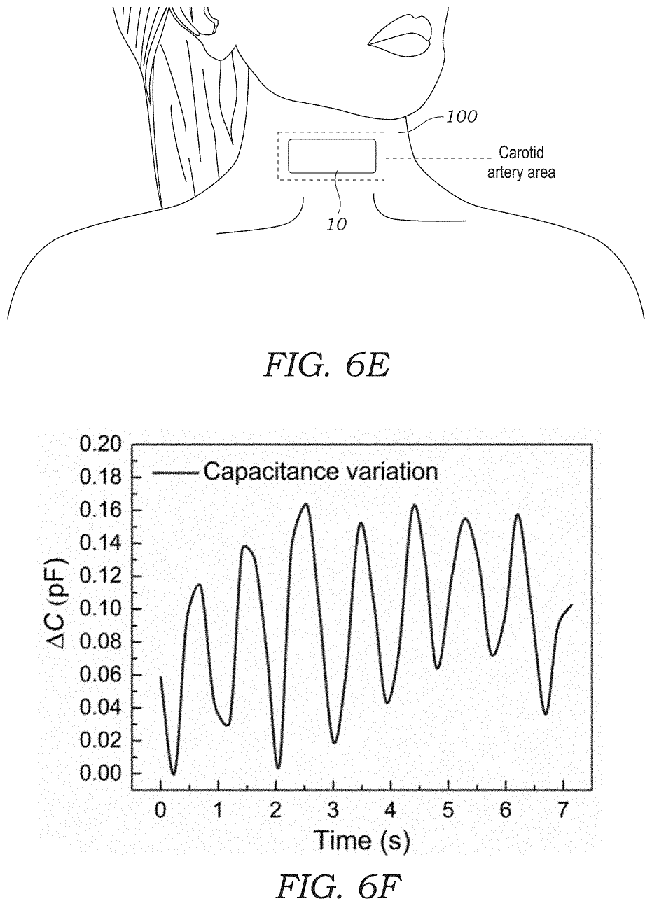

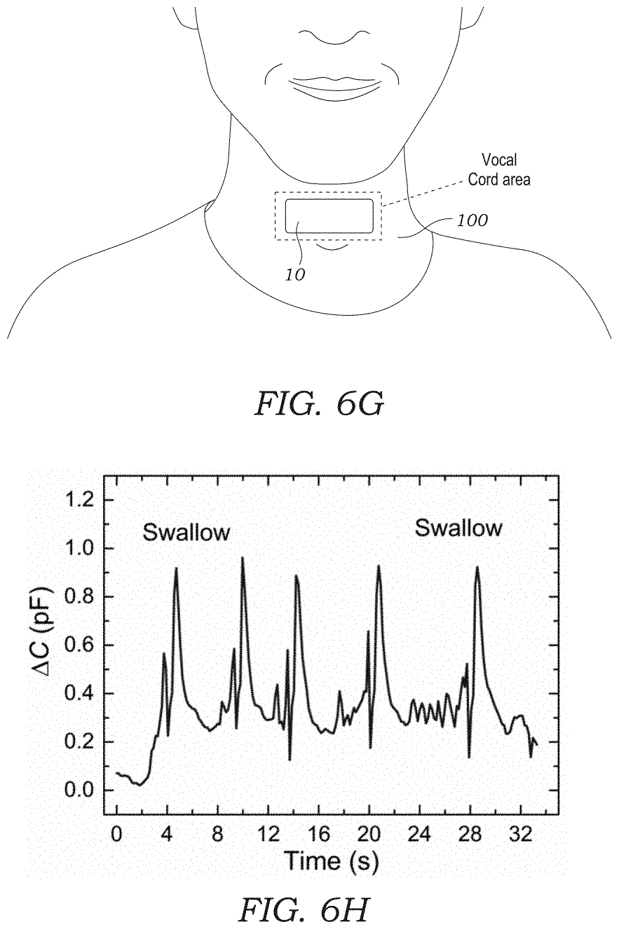

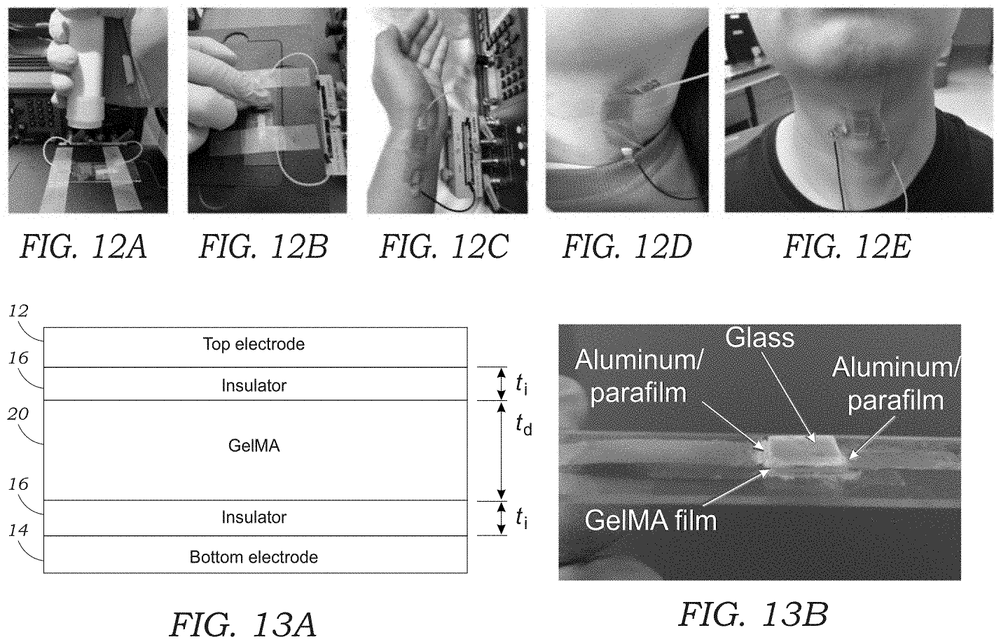

To demonstrate the potential of the GelMA tactile-based biosensor device 10 for practical applications, the biosensor device 10 was tested under air blowing pressure and finger touch (FIGS. 12A and 12B). The capacitance of the biosensor device 10 increased immediately in the presence of air pressure (˜0.1 kPa) and returned to its original value in the absence of the air pressure (FIG. 6A). Similarly, the biosensor device 10 was responsive to a gentle finger touch with a pressure of ˜1 kPa (FIG. 6B). In both cases, the base capacitance remained stable after cyclic tests, demonstrating the GelMA-based pressure sensors 10 have great potential to monitor the subtle pressure as wearable sensors.

The above results demonstrate the utility of the GelMA pressure sensor 10 and encourage its application in monitoring various human physiological signals. Toward this end, the biosensor device 10 was attached to different parts of the human body: the radial artery area on the wrist (FIG. 6C and FIG. 12C), and the carotid artery area on the neck (FIG. 6E and FIG. 12D). As shown in FIGS. 6C-6F, the capacitance of the pressure sensor 10 changed immediately in the presence of a pulse. Different heartbeats of 63 bpm (wrist) and 66 bpm (neck) were recorded from two volunteers. In particular, the pressure sensor 10 attached on the wrist can distinguish two radial artery pressure peaks, P₁ and P₂ from each pulse waveform (FIG. 6D), which are related to two crucial clinic parameters for arterial stiffness diagnosis: the radial augmentation index (A_{Ir}=P₂/P₁) and the time delay between P₁ and P₂ (ΔT_{DVP}=t₂−t₁). For the case tested, the A_{Ir} and ΔT_{DVP} are 66% and 486 ms, respectively, which is within the reasonable range of those of a volunteer. These results demonstrate that the GelMA-based wearable pressure sensors 10 are promising for healthcare applications that require real-time monitoring of physiological signals.

To further explore the potential of the GelMA biosensor device 10 for additional applications, the biosensor device 10 was attached to the larynx knot to examine its capability of detecting real-time swallowing and vocal cord vibration (FIG. 6G and FIG. 12E). In the swallowing test, the capacitance showed a quick response during each swallowing activity with stable base capacitance after multiple tests (FIG. 6H). In the vocal cord vibration test, the capacitance of the biosensor device 10 formed distinct shapes in response to different spoken words of “U”-C”-“L”-“A” (FIG. 6I). An identical capacitance shape was observed in the subsequent verification test, demonstrating the reliability of the biosensor device 10 and its potential for language recognition applications.

Synthesis of GelMA: GelMA was synthesized using the procedure as described previously, in Yue K. et al., Synthesis, properties, and biomedical applications of gelatin methacryloyl (GelMA) hydrogels. Biomaterials, 2015, 73(254-271), which is incorporated by reference herein. First, 10 g gelatin from porcine skin was added to 100 mL of Dulbecco's phosphate buffered saline (DPBS) (GIBCO) preheated at 50° C. The mixture was stirred at 50° C. until the gelatin was completely dissolved. Sequentially, a certain volume of mythacrylic anhydride (MA) (Sigma-Aldrich) was added to the gelatin solution, stirring at 50° C. to react for 2 hours. Then, the reaction was stopped by adding another preheated 100 mL of DPBS at 50° C. Next, the mixture solution was dialyzed at 40° C. using dialysis tubing (12-14 kDa) in distilled water for at least 5 days to remove methacrylic acid and other impurities such as salt. After dialysis, the resulting clear solution was freeze-dried for at least 5 days to form porous foam and stored at −80° C. for further use. Herein, four types of volumes of MA, 20 mL, 8 mL, 4 mL and 2 mL, were added to the 100 mL of gelatin solution to synthesize GelMA with different DS of MA. The DS for the synthesized GelMA with MA volume ratios of 20% (ultra-GelMA), 8% (high-GelMA), 4% (media-GelMA) and 2% (low-GelMA), are about 85%, 75%, 60% and 40%, respectively.

Preparation of GelMA hydrogel samples: GelMA hydrogel precursors were synthesized by dissolving solid GelMA into distilled ionic (DI) water at 80° C. for 20 minutes after the addition of photoinitiator (Irgacure® 2959). The GelMA hydrogel samples for electrical properties testing were prepared by pouring the warm GelMA hydrogel precursor into a 2 cm×2 cm×1 cm PDMS mold with a glass placed on the top and exposing it to a UV light of 45 mW/cm² for a given period. An 8 mm circular film was punched after UV crosslinking for further mechanical property testing. The GelMA hydrogel samples for the electrical properties testing were prepared directly with the PDMS mold.

Mechanical and electrical property characterization of GelMA hydrogels: The mechanical properties, such as the storage (elastic) modulus and loss (viscous) modulus, of GelMA hydrogel were tested using a Rheometer (MCR 301, Anton Paar). Oscillatory measurements were performed at 1% strain, constant frequency (1 Hz) and room temperature (25° C.). Twenty points were obtained for each sample, and the averaged value was used as the final result. For the electrical property characterization, the crosslinked 2 cm×2 cmxl cm hydrogel films were sandwiched by two self-made electrodes to form a parallel capacitor (see FIGS. 13A-13B for the sandwiched structure). A LCR meter 30 (E4980AL, Keysight Technologies) was used to measure its capacitances. The relative permittivity of GelMA hydrogel was calculated by the relationship of permittivity with capacitance (see equation (4)).

Preparation and testing of layer-by-layer stacked pressure sensors: Two types of simply stacked pressure sensors 10 were prepared: one with GelMA material 20 used as dielectric layers (FIG. 3A), and the reference one with PDMS as dielectric layers (FIG. 3B). As shown in FIG. 3A, the aluminum film was used as the electrodes 12, 14, and parafilm was used as the insulation layer 16 between the aluminum film and GelMA dielectric layer because GelMA hydrogel is electrically conductive. The aluminum film, parafilm, and middle dielectric layer were stacked together to form the pressure sensor 10. All dimensions of the GelMA-based pressure sensor 10 are the same as those of the PDMS-based pressure sensor. The thicknesses of the dielectric layer and parafilm are 0.7 mm and 0.085 mm, respectively. The area of each layer is the same as being: 10 mm×10 mm. The stacked pressure sensor 10 was tested at a preload of 4 kPa, which aimed to reduce the air gap between different layers and to make their structure compact. For each set of conditions, three sensors were tested, and the mean value was used as the final result.

GelMA-based dielectric layers were prepared by pouring heated GelMA hydrogel precursor into PDMS molds with a glass slide placed on the top and exposing them to UV light for 5 minutes. PDMS dielectric layers were made by spinning 10:1 (silicone base to the cure agent ratio) PDMS mixture on glass slides and curing at 80° C. oven for 2 hours.

Preparation of silicon mold: A 0.5 mm thick [100] silicon wafer with 100 nm thick Si₃N₄ on both sides was used to prepare the mold. Photolithography was performed after SPR 700 photoresist was spin-coated on top of the wafer for patterning. Reactive ion etching was then performed to remove the Si₃N₄ and expose the Si. Then the wafer was immersed in 30% potassium hydroxide (KOH) solution to etch away the exposed Si part with Si₃N₄ as the etching mask. Finally, the silicon wafer with recessed micro pyramidal structures was cleaned sequentially with acetone and alcohol for future use.

Fabrication of PDMS mold: The PDMS mold was made using a similar procedure as reported in Tee B. et al., Tunable Flexible Pressure Sensors using Microstructured Elastomer Geometries for Intuitive Electronics. Advanced Functional Materials, 2014, 24(34):5427-5434, which is incorporated herein by reference. A 5:1 mixture of PDMS base to crosslinker (Sylgard 184, Dow Corning) was mixed by adequately stirring and degassing in a vacuum chamber until all air bubbles disappeared. Next, the degassed mixture was poured on the silicon mold, degassed again, and cured at 80° C. for at least 4 hours. Sequentially, the cured PDMS was cut off to form the first inverted mold, and then treated with a spin-coated layer of cetyltrimethylammoniumbromide (CTAB) solution and dried in an 80° C. oven. Then, the final PDMS mold was made based on the first inverted mold with an identical procedure.

PEDOT:PSS mixture preparation: PEDOT:PSS (Clevios PH1000 from Heraeus Electronic Materials) solution was obtained by adding 5 v/v % of glycerol, 0.2 v/v % of 3-glycidoxypropyltrimethoxysilane (GOPS) and 1 v/v % of capstone. The mixture was sonicated for 20 minutes and then filtered with 0.45 μm syringe filters for further use.

Fabrication and characterization of PDMS/PEDOT:PSS/PDMS film: The fabrication process of PDMS/PEDOT:PSS/PDMS film started with a 10:1 mixture of PDMS base (Sylgard 184, Dow Corning) to crosslinker mixed by adequately stirring and degassing in a vacuum chamber until all air bubbles disappeared. The mixture was spin-coated on a pre-treated glass with CTAB solution at 2,000 rpm, and then cured at 80° C. for 2 hours (FIG. 10A). The cured PDMS film was cleaned with isopropanol (IPA) for 30 seconds, dried with a nitrogen gun, and then treated with oxygen plasma etch for 2 minutes. Immediately after, PEDOT:PSS solution was spin-coated on the PDMS film at 1,000 rpm, and cured on a hotplate at 160° C. for 50 minutes (FIG. 10B). Once cured, the copper lead wire 24 was attached to the PEDOT:PSS film with conductive sliver glue and annealed at 100° C. for 2 hours to reduce the contact resistance between them (not shown in the schematic). Next, another PDMS film was spin-coated on the PEDOT:PSS layer at 1500 rpm and cured at 80° C. for 2 hours again (FIG. 10C). Once cured, the PDMS/PEDOT:PSS/PDMS film was carefully peeled off from the glass slides; thoroughly cleaned with methanol, and completely dried with a nitrogen gun (FIG. 10D). It was further treated with benzophenone solution (10 w/v % in ethanol) for 5 minutes at room temperature, and washed with methanol and dried by nitrogen again for further bonding with microstructured GelMA hydrogel material 20 dielectric layer (FIG. 10F). The thicknesses of the PDMS/PEDOT:PSS/PDMS films are 40 um/10 um/60 um, respectively, which were tested using Dektak 150 Surface Profiler (Veeco Instruments Inc., USA).

Bonding of the GelMA hydrogel dielectric layer with the PDMS/PEDOT:PSS/PDMS film: During the fabrication process of the GelMA-based pressure sensors 10, two UV crosslinking steps were used. The first step to partially bond the GelMA hydrogel and PDMS was conducted by exposing the device to 45 mW/cm² of UV light for about 50 seconds for microstructured GelMA hydrogel dielectric layers and 30 seconds for the reference flat GelMA dielectric layer. The final crosslinking step was implemented by exposing the biosensor device 10 to UV light for 5 minutes under the same UV light strength. The exposed edges of the fabricated GelMA pressure sensors 10 were sealed with glue.

Performance testing of all solution-processed GelMA pressure sensors: An Instron (5900 Series) with a force resolution of 1×10⁻⁴ N was used to apply pressure. The force and displacement of the test head can be accurately controlled by a computer. When testing, a glass (10 mg) with a size of 10 mm×10 mm was placed on the tested sensor for uniform pressure. The capacitance was measured using LCR meter 30 (E4980AL, Keysight Technologies) at 100 kHz with a 1 V AC signal unless otherwise specified. The transparency of the GelMA pressure sensor 10 was tested by Hitachi U-4100 spectrophotometer with an integrating sphere equipped.

Institutional Review Board (IRB) Approval for Human Subject Testing

The conducted human subject experiments were performed in compliance with the protocols that have been approved by the IRB at the University of California, Los Angeles (IRB #17-000170). All subjects gave written informed consent before participation in the study. For all demonstrations on human skin, signed consent was obtained from the volunteer.

Equations

$$x = \frac{{Pd}_{0}}{{E}_{0}} , \qquad ( 1 )$$

$$\frac{Δ C}{{C}_{0}} = [ \frac{{ε}_{r} {ε}_{0} S}{( d - x )} - \frac{{ε}_{r} {ε}_{0} S}{d} ] / ( \frac{{ε}_{r} {ε}_{0} S}{d} ) = \frac{x}{( d - x )} = \frac{{Pd}_{0}}{( {r}_{d} {d}_{0} {E}_{0} - {Pd}_{0} )} ,$$

$$d = {r}_{d} {d}_{0}$$

$$x = \frac{{Pd}_{0}}{{E}_{0}} , \qquad ( 2 )$$

$$\frac{Δ C}{{C}_{0}} = [ \frac{{ε}_{r} {ε}_{0} S}{( d - x )} - \frac{{ε}_{r} {ε}_{0} S}{{d}_{0}} ] / ( \frac{{ε}_{r} {ε}_{0} S}{{d}_{0}} ) = \frac{{xPd}_{0}}{( d - x )} = \frac{{Pd}_{0}}{( {r}_{d} {d}_{0} {E}_{0} - {Pd}_{0} )} ,$$

$$E = {r}_{E} {E}_{0}$$

$$S = \frac{( Δ C / {C}_{0} )}{Δ P} \qquad ( 3 )$$

$${ε}_{d} = \frac{{t}_{d} {ε}_{i} C}{{ε}_{i} {ε}_{0} C - 2 {t}_{i} C} \qquad ( 4 )$$

Equation (4) presents the method to calculate the relative permittivity (dielectric constant) of GelMA hydrogel under different conditions. Herein, C represents the measured capacitance; and ε₁, ε_{d} and ε₀ are relative permittivities of the insulator, GelMA hydrogel and air, respectively. The relative permittivities of the parafilm and air are 2.2 and 1, respectively.

TABLE 1

| Ref. No. | |
|---|---|
| (FIG. 5C) | Citation |
| 17 | Lei Z. et al., A Bioinspired Mineral Hydrogel as a |
| | Self-Healable, Mechanically Adaptable Ionic Skin for |
| | Highly Sensitive Pressure Sensing. Advanced |
| | Materials, 2017, 29(22) |
| 18 | Ge G. et al., Stretchable, Transparent, and Self- |
| | Patterned Hydrogel-Based Pressure Sensor for |
| | Human Motions Detection. Advanced Functional |
| | Materials, 2018, 28(32) |
| 20 | Tai Y. et al., A highly sensitive, low-cost, wearable |
| | pressure sensor based on conductive hydrogel |
| | spheres. Nanoscale, 2015, 7(35): 14766-14773 |
| 25 | Wang Z. et al., Flexible and Washable Poly(Ionic |
| | Liquid) Nanofibrous Membrane with Moisture Proof |
| | Pressure Sensing for Real-Life Wearable Electronics. |
| | ACS Applied Materials & Interfaces, 2019, |
| | 11(30): 27200-27209 |
| 26 | Qin Z. et al., Carbon Nanotubes/Hydrophobically |
| | Associated Hydrogels as Ultrastretchable, Highly |
| | Sensitive, Stable Strain, and Pressure Sensors. ACS |
| | Applied Materials & Interfaces, 2020, 12(4): 4944- |
| | 4953 |
| 28 | Duan J. et al., Ultra-Stretchable and Force-Sensitive |
| | Hydrogels Reinforced with Chitosan Microspheres |
| | Embedded in Polymer Networks. Adv Mater, 2016, |
| | 28(36): 8037-8044 |

TABLE 2

| Literature | Height (μm) | Base width (μm) | spacing (μm) |
|---|---|---|---|
| Ref. [1] | 2 | 4 | 5.6 |
| Ref. [2] | 3 | 6 | 8.85 |
| Ref.[3] | 7.6 | 11.5 | — |
| Ref.[4] | 34 | 55 | 182 |
| Biosensor Device | 420 | 600 | 1000 |

[1] Boutry C. M., Nguyen A., Lawai Q. O. et al. A Sensitive and Biodegradable Pressure Sensor Array for Cardiovascular Monitoring. Adv Mater, 2015, 27(43): 6954-6961.
[2] Schwartz G., Tee B. C. K., Mei J. et al. Flexible polymer transistors with high pressure sensitivity for application in electronic skin and health monitoring. Nature Communications, 2013, 4(1).
[3]Shi R., Lou Z., Chen S. et al. Flexible and transparent capacitive pressure sensor with patterned microstructured composite rubber dielectric for wearable touch keyboard application. Science China Materials, 2018, 61 (12): 1587-1595.
[4]Tee B. C. K., Chortos A., Dunn R. R. et al. Tunable Flexible Pressure Sensors using Microstructured Elastomer Geometries for Intuitive Electronics. Advanced Functional Materials, 2014, 24(34): 5427-5434.

While embodiments of the present invention have been shown and described, various modifications may be made without departing from the scope of the present invention. While the biosensor device 10 described herein operates as a capacitor it may be easily used to make other types of soft sensors. Also, the insulator layers 16, 18 may be made from biodegradable proteins making the sensor fully biocompatible. The invention, therefore, should not be limited, except to the following claims, and their equivalents.

1. A tissue-compatible biosensor device comprising:
a first electrode comprising a biocompatible electrically conductive polymer;
an inner insulation layer disposed on an inner side of the first electrode;
a second electrode comprising a biocompatible electrically conductive polymer, the second electrode spaced apart from the first electrode to form a gap;
an inner insulation layer disposed on an inner side of the second electrode; and
crosslinked gelatin methacryloyl (GelMA) disposed in the gap between the inner side of the first electrode and the inner side of the second electrode.

2. The biosensor device of claim 1, wherein the gelatin methacryloyl (GelMA) disposed in the gap comprises a continuous layer of gelatin methacryloyl (GelMA) material.

3. The biosensor device of claim 1, wherein the gelatin methacryloyl (GelMA) disposed in the gap comprises a plurality of gelatin methacryloyl (GelMA) microstructures.

4. The biosensor device of claim 3, wherein the microstructures are shaped as pyramids, needles, hemispheres, cylinders, posts, fins, or grates.

5. The biosensor device of claim 4, wherein the microstructures have a height of less than 750 μm.

6. The biosensor device of claim 1, further comprising an outer layer disposed on an outer side of the first electrode and further comprising an outer layer disposed on an outer side of the second electrode.

7. The biosensor device of claim 6, wherein the outer layers and the inner insulation layers comprise polydimethylsiloxane (PDMS).

8. The biosensor device of claim 6, wherein the biosensor device is substantially optically transparent.

9. The biosensor device of claim 1, further comprising a first wire electrically connected to the first electrode and a second wire electrically connected to the second electrode.

10. The biosensor device of claim 1, wherein the first electrode and the second electrode comprise poly(3,4-ethylenedioxythiophene) polystyrene sulfonate (PEDOT:PSS).

11. The biosensor device of claim 1, further comprising a LCR meter or capacitance measuring circuitry electrically coupled to the first electrode and the second electrode.

12. A method of using the biosensor device of claim 1 comprising:
placing the biosensor device onto tissue; and
measuring a change in capacitance between the first electrode and the second electrode with a capacitance measurement device.

13. The method of claim 12, wherein the biosensor device is placed on skin tissue of a mammal.

14. The method of claim 13, wherein the biosensor device measures a pulse rate based on the change in capacitance.

15. The method of claim 13, wherein the biosensor device measures vocal cord vibrations based on the change in capacitance.

16. The method of claim 13, wherein the biosensor device measures swallowing based on the change in capacitance.

17. The method of claim 13, wherein the biosensor device measures respiration and/or respiration rate based on the change in capacitance.

18. The method of claim 13, wherein the biosensor device measures blood pressure based on the change in capacitance.

19. The method of claim 13, wherein the biosensor device measures and/or senses touch based on the change in capacitance.

20. A method of manufacturing a biosensor comprising:
a) providing a polydimethylsiloxane (PDMS) mold having a negative topology pattern formed thereon;
b) filling the PDMS mold with gelatin methacryloyl (GelMA) precursor;
c) laminating a first electrode structure to the filled PDMS mold and exposing the same to ultraviolet (UV) radiation to at least partially crosslink the GelMA and bond the first electrode structure;
d) removing the GelMA and bonded first electrode structure from the mold;
e) securing the removed GelMA and bonded first electrode structure to a second electrode structure; and
f) exposing the secured structure of (e) to UV radiation.

21. The method of claim 20, wherein the first electrode structure and the second electrode structure comprise a laminate of PDMS/PEDOT:PSS/PDMS.

22. The method of claim 21, wherein the laminate of PDMS/PEDOT:PSS/PDMS is treated in a solution of benzophenone.

23. The method of claim 20, further comprising securing wires to the first and second electrode structures.
